(19) United States
(12) Patent Application Publication
(10) Pub. No.: US 2015/0011912 A1
(43) Pub. Date: Jan. 8, 2015

(54) PERISTALTIC SOUND DETECTION APPARATUS, METHOD FOR DETECTING PERISTALTIC SOUND, AND RECORDING MEDIUM

(71) Applicants: Sharp Kabushiki Kaisha, Osaka-shi, Osaka (JP); University of Yamanashi, Kofu-shi, Yamanashi (JP)

(72) Inventors: Norihiro Matsuoka, Osaka-shi (JP); Kenichi Matsuda, Kofu-shi (JP); Osamu Sakata, Kofu-shi (JP); Norikazu Harii, Kofu-shi (JP)

(21) Appl. No.: 14/373,723

(22) PCT Filed: Jan. 25, 2013
(86) PCT No.: PCT/JP2013/051586

§ 371 Date: Jul. 22, 2014

(30) Foreign Application Priority Data
Jan. 25, 2012 (JP) 2012-013464

(51) Int. Cl. A61B 7/00; A61B 5/00

(52) U.S. Cl. CPC A61B 7/008; A61B 5/4255; A61B 5/7246
USPC 600/586

(57) ABSTRACT

A peristaltic sound detection apparatus (10) includes matching coefficient calculation means for calculating a plurality of matching coefficients by individually matching a frequency spectrum of a biological sound and a plurality of standard frequency spectra of peristaltic sounds.

### TECHNICAL FIELD

The present invention relates to a peristaltic sound detection apparatus, a method for detecting a peristaltic sound, a program, and a recording medium that determine whether or not a sound emitted by the intestines is a peristaltic sound.

### BACKGROUND ART

An evaluation apparatus and an evaluation method that quantitatively evaluate the activeness of digestive organs have not so far been put into practice. Therefore, whether or not digestive organs (for example, the intestines) are active is evaluated by a doctor. More specifically, the doctor hears peristaltic sounds of the abdomen using a stethoscope and evaluates whether or not the intestines are active on the basis of the peristaltic sounds. In other words, the activeness of the intestines is evaluated on the basis of the experience and subjectivity of the doctor, who is an evaluator.

Thus, when the activeness of digestive organs is evaluated, two steps, namely (i) detection of sounds emitted by the digestive organs and (ii) evaluation of the detected sounds through acoustic analyses, are taken.

In PTL 1, a technique for automatically detecting a time period in which a peristaltic sound has been recorded on the basis of a Fourier transform spectrum (frequency spectrum) obtained by Fourier transforming the peristaltic sound emitted by the digestive system is described. In this technique, a time period in the frequency spectrum having a peak of significant intensity within a range of 100 to 1,000 Hz is determined as a period in which a peristaltic sound has been recorded.

As an attempt to detect and analyze sounds emitted by a living body, in PTL 2, a speech recognition method is described in which a word or the like corresponding to an input sound is selected from a given dictionary. It is described that in this technique, DP (dynamic programming) matching can be used when a corresponding word is selected. In the DP matching, standard patterns corresponding to words that are targets of the speech recognition are created in advance. A feature quantity obtained as a result of the acoustic analyses of an input sound and the standard patterns are matched, and, for example, a word corresponding to a standard pattern that is most similar to the feature quantity of the sound is determined as the result of the speech recognition. When a detected sound pattern and a comparative sound pattern (standard pattern) are subjected to the pattern matching, the number of standard patterns is generally one.

In PTL 3, a technique is described in which Wigner distribution is adopted as the frequency distribution of valve acoustic waveforms detected by a phonocardiograph when the valves are closed, and then the first-order moment is obtained from the Wigner distribution for each time.

A biological sound detection process apparatus described in PTL 4 performs an FFT process on biological sound detection data (respiratory sound detection data) to calculate an amplitude spectrum, a phase spectrum, and a power spectrum. Furthermore, the biological sound detection process apparatus calculates a local average value and a local dispersion value from the power spectrum. In accordance with the magnitude of the local dispersion value, the amplitude spectrum is classified into one corresponding to a normal respiratory sound or one caused by continuous sounds.

A cough detection apparatus described in PTL 5 extracts sound signals in first and second frequency bands from sound signals obtained through a direct contact microphone. The cough detection apparatus determines candidate coughs from the first band signals. Furthermore, the cough detection apparatus determines whether or not the candidate coughs are actual coughs on the basis of correspondence between the candidate coughs and the second band signals.

### CITATION LIST

### Patent Literature

PTL 1: Japanese Unexamined Patent Application Publication No. 2000-262523 (Sep. 26, 2000)

PTL 2: Japanese Unexamined Patent Application Publication No. 9-160585 (Jun. 20, 1997)

PTL 3: Japanese Unexamined Patent Application Publication No. 6-90913 (Apr. 5, 1994)

PTL 4: Japanese Unexamined Patent Application Publication No. 2004-357758 (Dec. 24, 2004)

PTL 5: Japanese Unexamined Patent Application Publication No. 2009-233103 (Oct. 15, 2009)

### SUMMARY OF INVENTION

### Technical Problem

There are a plurality of occurrence modes of peristaltic movement in the intestines. In addition, biological sounds emitted by the intestines during peristaltic movement vary among individuals. For this reason, the frequency spectra of the biological sounds can significantly vary.

Therefore, in the technique disclosed in PTL 1, in which whether or not a biological sound is a peristaltic sound is determined on the basis of whether or not there is a peak of significant intensity within a particular range of the frequency spectrum, it is difficult to accurately detect a peristaltic sound. In addition, in the pattern matching between a detected sound pattern and a standard pattern, which is the technique described in PTL 2, too, it is difficult to accurately evaluate a desired sound.

On the other hand, in the techniques described in PTL 3 to PTL 5, complex arithmetic processing is performed for the acoustic analyses. Therefore, there is a problem in that the time taken for the acoustic analyses to be completed is long. In addition, there is also a problem in that a high-performance processing apparatus is required in order to perform complex processing. That is, in the techniques described in PTL 3 to PTL 5, there are problems in that the apparatuses used are costly and large in size and it takes a long time to obtain results of the acoustic analyses.

The present invention has been established in view of the above problems. An object of the present invention is to provide a peristaltic sound detection apparatus and a method for detecting a peristaltic sound that are capable of accurately determining whether or not a sound emitted by the intestines is a peristaltic sound without performing complex arithmetic processing to analyze the sound.

### Solution to Problem

In order to solve the above problems, a peristaltic sound detection apparatus according to an aspect of the present invention includes biological sound detection means for detecting a biological sound emitted by intestines, frequency spectrum calculation means for calculating a frequency spectrum of the biological sound, matching coefficient calculation means for calculating a plurality of matching coefficients by individually matching the frequency spectrum of the biological sound and a plurality of standard frequency spectra of peristaltic sounds, and peristaltic sound determination means for determining whether or not the biological sound is a peristaltic sound by performing arithmetic processing on the plurality of matching coefficients.

In order to solve the above problems, a method for detecting a peristaltic sound according to an aspect of the present invention includes a biological sound detection step of detecting a biological sound emitted by intestines, a frequency spectrum calculation step of calculating a frequency spectrum of the biological sound, a matching coefficient calculation step of calculating a plurality of matching coefficients by individually matching the frequency spectrum of the biological sound and a plurality of standard frequency spectra of peristaltic sounds, and a peristaltic sound determination step of determining whether or not the biological sound is a peristaltic sound by performing arithmetic processing on the plurality of matching coefficients.

Other objects, characteristics, and advantages of the present invention will be sufficiently clarified by the following description. In addition, benefits of the present invention will become apparent from the following description with reference to the attached drawings.

### Advantageous Effects of Invention

According to a peristaltic sound detection apparatus and a method for detecting a peristaltic sound according to aspects of the present invention, the accuracy of determining whether or not a sound emitted by the intestines is a peristaltic sound can be improved without performing complex arithmetic processing to analyze the sound.

### BRIEF DESCRIPTION OF DRAWINGS

FIG. 1 is a block diagram illustrating the configuration of a peristaltic sound detection apparatus according to an embodiment of the present invention.

FIG. 2 is a flowchart illustrating a procedure performed by the peristaltic sound detection apparatus according to the embodiment of the present invention for determining whether or not a biological sound is a peristaltic sound.

FIG. 3 is a diagram illustrating standard frequency spectra used when the peristaltic sound detection apparatus according to the embodiment of the present invention calculates matching coefficients. (a) illustrates a first standard frequency spectrum, (b) illustrates a second standard frequency spectrum, and (c) illustrates a third standard frequency spectrum.

FIG. 4 is a diagram illustrating detection rates of peristaltic sounds of the peristaltic sound detection apparatus in an example of the present invention and the peristaltic sound detection apparatus in a comparative example.

FIG. 5 is a diagram illustrating a result obtained by observing the intestinal peristaltic sound occurrence frequency of a subject administered midozolam, buprenorphine, and propofol using the peristaltic sound detection apparatus according to the embodiment of the present invention.

FIG. 6 is a diagram illustrating a result obtained by observing the intestinal peristaltic sound occurrence frequency of a subject administered propofol and DEX using the peristaltic sound detection apparatus according to the embodiment of the present invention.

### DESCRIPTION OF EMBODIMENTS

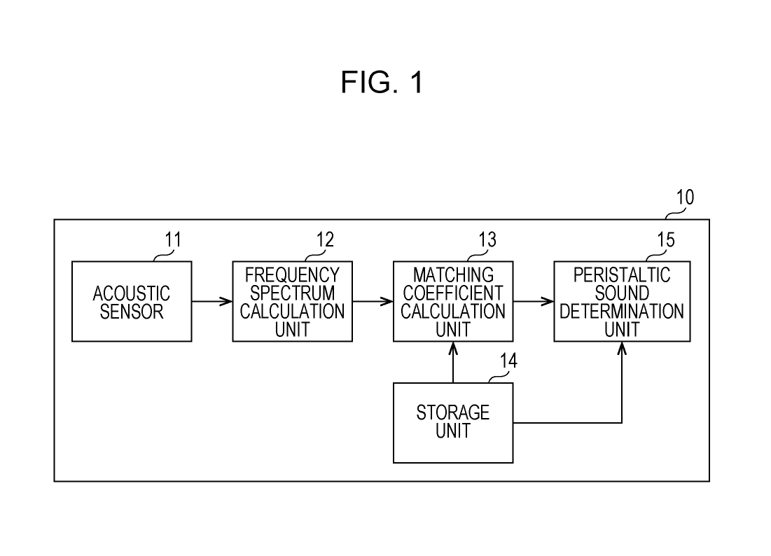

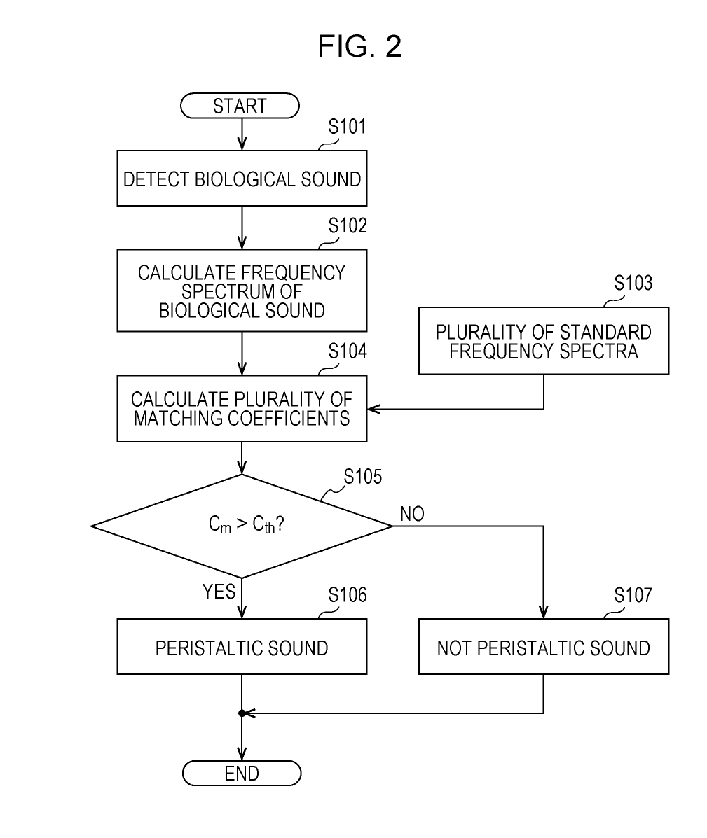

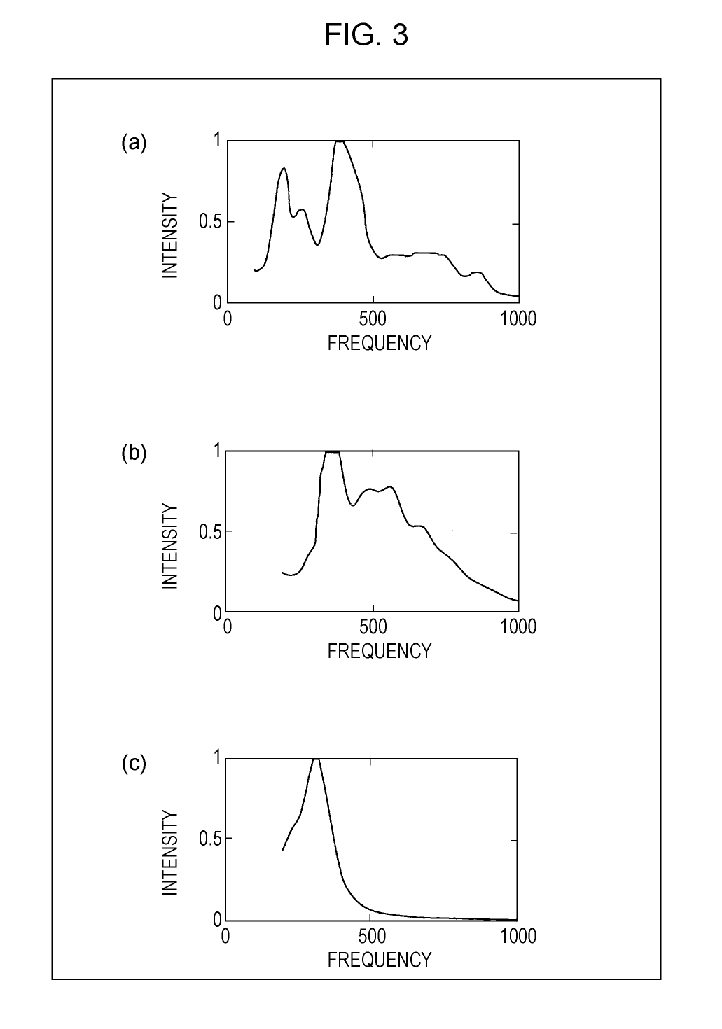

A peristaltic sound detection apparatus 10 according to an embodiment of the present invention will be described with reference to FIGS. 1 to 3. FIG. 1 is a block diagram illustrating the configuration of the peristaltic sound detection apparatus 10. FIG. 2 is a flowchart illustrating a procedure performed by the peristaltic sound detection apparatus 10 for determining whether or not a biological sound emitted by the intestines is a peristaltic sound. FIG. 3 is a diagram illustrating first to third standard frequency spectra used when the peristaltic sound detection apparatus 10 calculates matching coefficients.

As illustrated in FIG. 1, the peristaltic sound detection apparatus 10 includes an acoustic sensor 11, a frequency spectrum calculation unit 12, a matching coefficient calculation unit 13, a storage unit 14, and a peristaltic sound determination unit 15.

The peristaltic sound detection apparatus 10 is an apparatus that determines whether or not a biological sound emitted by the intestines is a peristaltic sound by detecting and analyzing the biological sound emitted by the intestines. In other words, the peristaltic sound detection apparatus 10 is an apparatus that detects a biological sound emitted by the intestines. In the following description, a “biological sound emitted by the intestines” will also be referred to simply as a “biological sound”.

The components of the peristaltic sound detection apparatus 10 will be described hereinafter with reference to FIGS. 1 and 3. The procedure performed by the peristaltic sound detection apparatus 10 for determining whether or not a biological sound is a peristaltic sound will be described later with reference to the flowchart of FIG. 2.

(Acoustic Sensor 11)

The acoustic sensor 11, which is biological sound detection means, converts a detected sound signal into an electrical signal. As the acoustic sensor 11, for example, a direct contact acoustic microphone may be used. The acoustic sensor 11 may be fixed by a person who operates the peristaltic sound detection apparatus 10 at a position at which biological sounds of a subject can be detected. The number of acoustic sensors 11 that detect biological sounds is not limited, that is, the number of acoustic sensors 11 may be one or more. The acoustic sensor 11 outputs a detected biological sound to the frequency spectrum calculation unit 12 as an electrical signal.

The acoustic sensor 11 may include an amplifier (not illustrated) for amplifying an electrical signal. It is to be noted that the amplifier need not necessarily be included in the acoustic sensor 11, but may be included in the frequency spectrum calculation unit 12.

(Frequency Spectrum Calculation Unit 12)

The frequency spectrum calculation unit 12, which is frequency spectrum calculation means, preferably includes an analog-to-digital conversion section (A/D conversion section). The A/D conversion section receives an electrical signal from the acoustic sensor 11 and converts the electrical signal into biological sound data, which is digital data.

Next, the frequency spectrum calculation unit 12 performs a fast Fourier transformation (FFT) process on the biological sound data at certain time intervals. In the following description, the certain time intervals will also be referred to as FFT process intervals. The FFT process intervals are preferably within a range of 0.3 to 1.0 second. More preferably, the FFT process intervals are within a range of 0.3 to 0.5 second.

If the FFT process intervals are set to be long, a plurality of intestinal peristaltic sounds might be undesirably included in one of the FFT process intervals. In other words, a plurality of intestinal peristaltic sounds might be detected as one intestinal peristaltic sound by mistake. In order to prevent such missed detection of intestinal peristaltic sounds, the FFT process intervals are preferably shorter than or equal to 1.0 second and, more preferably, shorter than or equal to 0.5 second.

On the other hand, as the set FFT process intervals get shorter, the execution frequency of the FFT process increases. That is, as the number of FFT processes executed increases, a calculation load upon the frequency spectrum calculation unit 12 increases. As a result, a real-time process, which will be described later, might become difficult to perform. Therefore, the FFT process intervals are preferably 0.3 second or longer.

It is to be noted that a lower limit value of the FFT process intervals depends on the arithmetic performance of the frequency spectrum calculation unit 12. When the arithmetic performance of the frequency spectrum calculation unit 12 is sufficiently high, the real-time process can be realized even if the FFT process intervals are set to be shorter than 0.3 second. That is, the lower limit value of the FFT process time is not limited to 0.3 second.

In this embodiment, the FFT process intervals are set to 0.32 second. That is, the frequency spectrum calculation unit 12 divides temporally continuous biological sound data into pieces of biological sound data of intervals of 0.32 second. Thereafter, the obtained pieces of biological sound data are sequentially subjected to the FFT process.

Any of known methods may be selected and used as a method for performing the FFT process. In this embodiment, the frequency spectrum calculation unit 12 executes the FFT process using a function “spectrogram” incorporated into MATLAB (registered trademark) developed by MathWorks. The frequency spectrum calculation unit 12 calculates the frequency spectrum (hereinafter also referred to as a biological sound spectrum) of the biological sound data by performing the FFT process on the biological sound data. More specifically, the frequency spectrum calculation unit 12 calculates one biological sound spectrum by performing the FFT process on each piece of biological data of 0.32 second.

The frequency spectrum calculation unit 12 sequentially outputs biological sound spectra calculated at the certain intervals to the matching coefficient calculation unit 13. It is to be noted that, in the following description, one of the biological sound spectra calculated at the certain intervals will be focused upon and described in order to clarify the description of each component.

(Standard Frequency Spectra)

The matching coefficient calculation unit 13, which will be described later, receives the biological sound spectrum from the frequency spectrum calculation unit 12 and reads standard frequency spectra to be matched with the biological sound spectrum from the storage unit 14. At this time, the matching coefficient calculation unit 13 reads a plurality of standard frequency spectra from the storage unit 14. In this embodiment, a plurality of biological sounds (hereinafter referred to as standard peristaltic sounds) that have been determined by a doctor as peristaltic sounds are extracted in advance, and these standard peristaltic sounds are subjected to the FFT process and stored in the storage unit 14 as the standard frequency spectra.

There are a plurality of occurrence modes of peristaltic sounds that occur during peristaltic movement of the intestines. The intestines emit different peristaltic sounds in accordance with the occurrence modes. Therefore, the standard frequency spectra used for the matching are preferably ones obtained by performing the FFT process on peristaltic sounds that occur in the plurality of different occurrence modes.

Because peristaltic sounds significantly vary from person to person, the plurality of standard frequency spectra may be obtained by performing the FFT process on a plurality of standard peristaltic sounds extracted from a plurality of persons. Alternatively, the standard frequency spectra may be obtained by performing the FFT process on a plurality of standard peristaltic sounds and extracting common spectra. Furthermore, the standard frequency spectra may be calculated by performing the FFT process on biological sounds emitted when the intestinal conditions are favorable and statistically processing the biological sounds subjected to the FFT process. It is to be noted that the plurality of standard frequency spectra may be configured by combining standard frequency spectra obtained from standard peristaltic sounds that occur in different occurrence modes and standard frequency spectra obtained from standard peristaltic sounds extracted from a plurality of persons.

In this embodiment, a first standard frequency spectrum (FIG. 3(a)), a second standard frequency spectrum (FIG. 3(b)), and a third standard frequency spectrum (FIG. 3(c)) are used as the standard frequency spectra for comparison references.

It is to be noted that the standard frequency spectra used for the matching are not limited to those illustrated in FIGS. 3(a) to 3(c). That is, two standard frequency spectra or four or more standard frequency spectra may be used for the matching.

(Matching Coefficient Calculation Unit 13)

The matching coefficient calculation unit 13, which is matching coefficient calculation means, calculates a plurality of matching coefficients (correlation coefficients) by matching (also referred to as seeing correlation, comparing, or extracting matching points) the biological sound spectrum and the standard frequency spectra.

More specifically, the matching coefficient calculation unit 13 calculates a first matching coefficient C₁ by matching the biological sound spectrum and the first standard frequency spectrum. Similarly, the matching coefficient calculation unit 13 calculates a second matching coefficient C₂ by matching the biological sound spectrum and the second standard frequency spectrum and a third matching coefficient C₃ by matching the biological sound spectrum and the third standard frequency spectrum.

A matching coefficient is a coefficient indicating the degree of similarity between a biological sound spectrum and a standard frequency spectrum. In other words, a matching coefficient is a coefficient indicating the number of elements of a standard frequency spectrum included in a biological sound spectrum. Therefore, as the matching coefficient becomes larger, a biological sound spectrum and a standard frequency spectrum are more similar, that is, the biological sound spectrum includes more elements of the standard frequency spectrum.

A known method may be used for the matching between the biological sound spectrum and the standard frequency spectra. In this embodiment, the matching coefficient calculation unit 13 calculates the matching coefficients using a function “corrcoef” incorporated into MATLAB (registered trademark) developed by MathWorks.

The matching coefficient calculation unit 13 may be configured to calculate a matching coefficient that is a real number within a range of 0 to 1. If the matching coefficient calculation unit 13 is configured in this way, a matching coefficient of 1 indicates that a peristaltic sound spectrum and a standard spectrum match.

The matching coefficient calculation unit 13 outputs the plurality of matching coefficients C₁, C₂, and C₃ calculated as a result of the matching to the peristaltic sound determination unit 15.

If a biological sound emitted by the intestines includes an element of a peristaltic sound that occurs in at least one of the plurality of occurrence modes, the peristaltic sound detection apparatus 10 can detects the element of the peristaltic sound as at least one of C₁, C₂, and C₃. In other words, even if a biological sound is configured by a combination of peristaltic sounds that occur in a plurality of occurrence modes, the peristaltic sound detection apparatus 10 can individually detect elements of these peristaltic sounds. Therefore, it is possible to reduce missed detection of peristaltic sounds and accurately determine whether or not a biological sound is a peristaltic sound.

It is to be noted that if peristaltic sounds extracted from a plurality of persons are subjected to the FFT process and used as a plurality of standard frequency spectra, missed detection of peristaltic movement caused by differences in peristaltic sound among individuals can be reduced.

(Peristaltic Sound Determination Unit 15)

The peristaltic sound determination unit 15, which is peristaltic sound determination means, calculates a determination matching coefficient Cₘ by performing arithmetic processing on the plurality of matching coefficients C₁, C₂, and C₃ received from the matching coefficient calculation unit 13. More specifically, the peristaltic sound determination unit 15 performs processing for comparing the values of C₁, C₂, and C₃ and determines the matching coefficient C₁, C₂, or C₃, whichever is the largest, as the determination matching coefficient Cₘ.

Furthermore, the peristaltic sound determination unit 15 compares the values of the determination matching coefficient Cₘ and a certain threshold Cₜₕ. If a result is Cₘ>Cₜₕ, the peristaltic sound determination unit 15 determines that the biological sound is a peristaltic sound, and if the result is Cₘ≦Cₜₕ, the peristaltic sound determination unit 15 determines that the biological sound is not a peristaltic sound.

The threshold Cₜₕ may be any positive real number, and empirically it is preferable to set the threshold Cₜₕ within a range of 0.5≦Cₜₕ≦0.9. For example, the threshold Cₜₕ may be 0.8. If the threshold Cₜₕ is set to be large, a stricter criterion is set for the determination of a peristaltic sound. On the other hand, if the threshold Cₜₕ is set to be small, a looser criterion is set for the determination of a peristaltic sound. In other words, if the threshold Cₜₕ is too large, the possibility of erroneous detection in which an intestinal peristaltic sound is not detected as an intestinal peristaltic sound becomes high, and if the threshold Cₜₕ is too small, the possibility of erroneous detection in which a biological sound other than an intestinal peristaltic sound is detected as an intestinal sound becomes high.

It is to be noted that the value of the threshold Cₜₕ with which an intestinal peristaltic sound can be properly detected depends on standard frequency spectra used for the matching, that is, more specifically, a combination of a plurality of standard frequency spectra. Therefore, an optimal value of the threshold Cₜₕ is experimentally set in accordance with the combination of a plurality of standard frequency spectra used for the matching.

The threshold Cₜₕ may be stored in the storage unit 14 in advance and read by the peristaltic sound determination unit 15 as necessary. The peristaltic sound detection apparatus 10 may be configured to be able to arbitrarily change the threshold Cₜₕ in accordance with an operation performed from the outside. Alternatively, the peristaltic sound determination unit 15 may include a ROM (read-only memory) that is not illustrated in FIG. 1, and the threshold Cₜₕ may be stored in the ROM in advance.

(Real-Time Process)

In the above description, one of the peristaltic sound spectra has been focused upon and described in order to clarify the operation of each component. In practice, as described with reference to the frequency spectrum calculation unit 12, the frequency spectrum calculation unit 12 continuously calculates peristaltic sound spectra at the certain time intervals.

Furthermore, the processes performed by the matching coefficient calculation unit 13 and the peristaltic sound determination unit 15 do not include complex analysis processes and accordingly can be processed in a short time relative to the certain time intervals. In other words, the processes performed by the matching coefficient calculation unit 13 and the peristaltic sound determination unit 15 are not processes that cause a delay in the determination as to whether or not a biological sound is a peristaltic sound.

Therefore, the peristaltic sound detection apparatus 10 can processes the series of processes, namely the detection of a peristaltic sound, the calculation of a peristaltic sound spectrum, the calculation of a plurality of matching coefficients, and the determination as to whether or not a biological sound is a peristaltic sound based on a comparison between the determination matching coefficient Cₘ and the threshold Cₜₕ, in real-time.

(Detection of Number of Peristaltic Sounds)

The peristaltic sound detection apparatus 10 can also detect the number of peristaltic sounds emitted by the intestines in unit time. More specifically, the peristaltic sound detection apparatus 10 may count the number of times that the peristaltic sound determination unit 15 determines a biological sound as a peristaltic sound in a certain unit time.

Since the peristaltic sound detection apparatus 10 can accurately determine whether or not a biological sound is a peristaltic sound, it is possible to avoid redundant counting of peristaltic sounds in unit time. In addition, the peristaltic sound detection apparatus 10 can check a change in intestinal activity by calculating the number of peristaltic sounds detected in unit time. The intestinal activity herein refers to both intestinal activity caused by ingested substances and intestinal activity that occurs regardless of ingested substances.

By keep counting the number of peristaltic sounds emitted by the intestines in unit time, the peristaltic sound detection apparatus 10 can be used for observing the activity of the intestines.

(Modification of Peristaltic Sound Determination Unit 15)

Here, a peristaltic sound determination unit 15′, which is a modification of the peristaltic sound determination unit 15, will be described.

Compared to the peristaltic sound determination unit 15, the peristaltic sound determination unit 15′ uses a different method for calculating the determination matching coefficient Cₘ. More specifically, the peristaltic sound determination unit 15′ calculates a value obtained by summing all the first to third matching coefficients C₁, C₂, and C₃ as the determination matching coefficient Cₘ.

Arithmetic processing for determining whether or not a biological sound is a peristaltic sound is the same as that performed by the peristaltic sound determination unit 15.

As described above, there are a plurality of occurrence modes of the peristaltic movement of the intestines. The peristaltic movement does not necessarily occur in a single occurrence mode, but might occur while including little bits of the elements of a plurality of occurrence modes. When the peristaltic sound detection apparatus 10 includes the peristaltic sound determination unit 15′, a biological sound can be detected as a peristaltic sound even if the biological sound includes little bits of the elements of a plurality of occurrence modes. Therefore, the accuracy of determining whether or not a biological sound is a peristaltic sound can be improved.

A certain threshold Cₜₕ′ used by the peristaltic sound detection apparatus 10 including the peristaltic sound determination unit 15′ may be any positive real number, and empirically it is preferable to set the threshold Cₜₕ′ within a range of 1≦Cₜₕ′≦1.8. For example, the threshold Cₜₕ′ may be 1.25.

As with the threshold Cₜₕ used by the peristaltic sound determination unit 15, if the threshold Cₜₕ′ is too large, the possibility of erroneous detection in which an intestinal peristaltic sound is not detected as an intestinal peristaltic sound becomes high, and if the threshold Cₜₕ′ is too small, the possibility of erroneous detection in which a biological sound other than an intestinal peristaltic sound is detected as an intestinal sound becomes high. In addition, as with the threshold Cₜₕ, it is preferable to experimentally set an optimal value of the threshold Cₜₕ′ in accordance with the combination of a plurality of standard frequency spectra used for the matching.

(Procedure for Detecting Peristaltic Sound)

A method for determining whether or not a biological sound is a peristaltic sound used by the peristaltic sound detection apparatus 10 will be described with reference to the flowchart of FIG. 2.

(S101)

The peristaltic sound detection apparatus 10 detects a biological sound through the acoustic sensor 11 and outputs the biological sound to the frequency spectrum calculation unit 12 as an electrical signal (biological sound detection step).

(S102)

The frequency spectrum calculation unit 12 calculates the frequency spectrum of the biological sound (biological sound spectrum) by performing the FFT process on peristaltic sound data at the certain time intervals (frequency spectrum calculation step).

(S103)

In the storage unit 14, standard frequency spectra, which are the frequency spectra of standard peristaltic sounds that occur in a plurality of emission modes, are stored in advance. In this embodiment, the storage unit 14 stores the three standard frequency spectra. The standard frequency spectra corresponding to the occurrence modes are referred to as the first standard frequency spectrum, the second standard frequency spectrum, and the third standard frequency spectrum.

(S104)

The matching coefficient calculation unit 13 receives the biological sound spectrum from the frequency spectrum calculation unit 12 and reads the first to third standard frequency spectra from the storage unit 14. The matching coefficient calculation unit 13 calculates the first matching coefficient C₁ by matching the biological sound spectrum and the first standard frequency spectrum. Similarly, the matching coefficient calculation unit 13 calculates the second matching coefficient C₂ by matching the biological sound spectrum and the second standard frequency spectrum and the third matching coefficient C₃ by performing processing for comparing the biological sound spectrum and the third standard frequency spectrum (matching coefficient calculation step).

(S105)

The peristaltic sound determination unit 15 receives the first to third matching coefficients C₁, C₂, and C₃ from the matching coefficient calculation unit 13. The peristaltic sound determination unit 15 performs processing for comparing C₁, C₂, and C₃ and calculates the matching coefficient C₁, C₂, or C₃, whichever is the largest, as the determination matching coefficient Cₘ.

Furthermore, the peristaltic sound determination unit 15 compares the values of the determination matching coefficient Cₘ and the certain threshold Cₜₕ (peristaltic sound determination step).

(S106)

If the determination matching coefficient Cₘ is larger than the threshold Cₜₕ (YES in S105), the peristaltic sound determination unit 15 determines that the biological sound is a peristaltic sound (peristaltic sound determination step).

(S107)

On the other hand, if the determination matching coefficient Cₘ is smaller than or equal to the threshold Cₜₕ (NO in S105), the peristaltic sound determination unit 15 determines that the biological sound is not a peristaltic sound (matching coefficient determination step).

By determining whether or not a biological sound is a peristaltic sound using the above steps, the method for detecting a peristaltic sound according to an embodiment of the present invention produces an effect of improving the accuracy of the determination without performing complex arithmetic processing.

### FIRST EXAMPLE

Whether or not a biological sound is a peristaltic sound was determined using the peristaltic sound detection apparatus 10. It is to be noted that the peristaltic sound detection apparatus 10 used in a first example included peristaltic sound determination unit 15. That is, the matching coefficient C₁, C₂, or C₃, whichever was the largest, was used as the determination matching coefficient Cₘ.

Conditions used for determining the activeness of the intestines were as follows:
A direct contact acoustic microphone was used as the acoustic sensor 11 and affixed to a subject's abdomen.
The certain time intervals when the frequency spectrum calculation unit 12 performed the FFT process on biological sound data were 0.32 second.
As the standard frequency spectra used by the matching coefficient calculation unit 13, the first standard frequency spectrum (FIG. 3(a)), the second standard frequency spectrum (FIG. 3(b)), and the third standard frequency spectrum (FIG. 3(c)) were used.
The certain threshold Cₜₕ used by the peristaltic sound determination unit 15 was 0.8.
Detection of peristaltic sounds was continuously performed for 30 seconds.

Under such conditions, the peristaltic sound detection apparatus 10 determined whether or not a biological sound is a peristaltic sound, and a doctor also made determinations. As a result, a ratio of the number of biological sounds determined by the peristaltic sound detection apparatus 10 as “peristaltic sounds” to the number of biological sounds determined by the doctor as “peristaltic sounds” was 98%. The ratio will be referred to as a detection rate hereinafter.

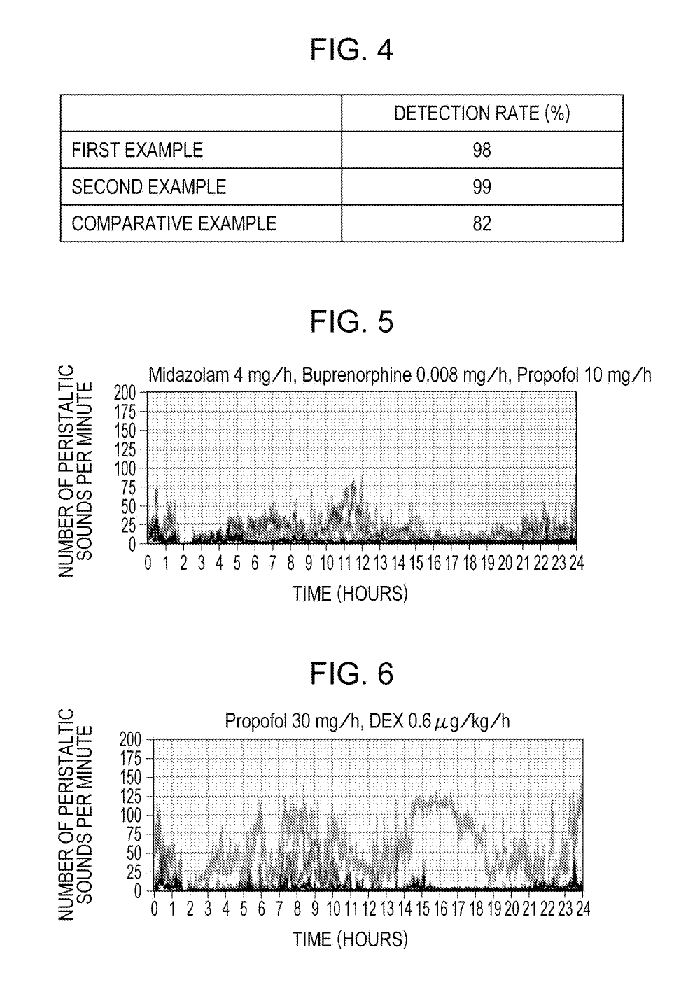

A table indicating the detection rate in the first example and detection rates in a second example and a comparative example, which will be described later, is illustrated in FIG. 4.

### SECOND EXAMPLE

Whether or not a biological sound is a peristaltic sound was determined using the peristaltic sound detection apparatus 10. It is to be noted that the peristaltic sound detection apparatus 10 used in a first example included the peristaltic sound determination unit 15′. That is, a value obtained by summing all of C₁, C₂, and C₃ was used as the matching coefficient Cₘ.

Conditions used for determining the activeness of the intestines were the same as those used in the first example except for the value of the certain threshold Cₜₕ′. In the second example, the threshold Cₜₕ′ was 1.25.

As a result, the detection rate obtained in the second example was 99%.

### COMPARATIVE EXAMPLE

As a comparative example of the first and second examples, peristaltic sounds were detected using one standard frequency spectrum. Whereas the first to third standard frequency spectra were used in the first and second examples, only the first standard frequency spectrum (FIG. 3(a)) was used in the comparative example. Other conditions are the same as those used in the first example.

As a result, the detection rate obtained in the comparative example was 82%.

As described above, compared to when whether or not a biological sound is a peristaltic sound was determined using one standard frequency spectrum, the detection rate significantly improved in the first and second examples, in which the first to third standard frequency spectra were used. That is, the accuracy of the determination improved by determining whether or not a biological sound is a peristaltic sound using a plurality of standard frequency spectra.

### THIRD EXAMPLE

Effects of medicines taken by a subject upon the digestive activity of the intestines were investigated. More specifically, the number of times that the peristaltic sound detection apparatus 10 determined a biological sound as a “peristaltic sound” in one minute was calculated as the number of intestinal peristaltic sounds occurred.

In this example, the peristaltic sound detection apparatus 10 included the peristaltic sound determination unit 15′. That is, a value obtained by summing all of C₁, C₂, and C₃ was used as the determination matching coefficient Cₘ. Conditions used for determining the activeness of the intestines were as follows:
Four direct contact acoustic microphones were used as acoustic sensors 11 and affixed to the subject's abdomen.
The certain time intervals when the frequency spectrum calculation unit 12 performed the FFT process on biological sound data were 0.32 second.
As the standard frequency spectra used by the matching coefficient calculation unit 13, the first standard frequency spectrum (FIG. 3(a)), the second standard frequency spectrum (FIG. 3(b)), and the third standard frequency spectrum (FIG. 3(c)) were used.
The certain threshold Cₜₕ′ used by the peristaltic sound determination unit 15 was 1.5.
Detection of peristaltic sounds was continuously performed for 24 hours, and the number of intestinal peristaltic sounds occurred per minute was calculated.

First, the peristaltic sound detection apparatus 10 detected intestinal peristaltic sounds while 4 mg/h of midazolam, 0.008 mg/h of buprenorphine, and 10 mg/h of propofol were being administered to the subject as sedatives and analgesics. The number of intestinal peristaltic sounds occurred per minute calculated from obtained results is illustrated in FIG. 5.

On the next day, the peristaltic sound detection apparatus 10 detected intestinal peristaltic sounds while 30 mg/h of propofol and 0.6 μg/kg/h of DEX were being administered to the same subject as sedatives. The number of intestinal peristaltic sounds occurred per minute calculated from the number of intestinal peristaltic sounds detected is illustrated in FIG. 6.

By comparing FIG. 5 and FIG. 6, it can be seen that the number of intestinal peristaltic sounds occurred is larger in FIG. 6. That is, results were obtained indicating that the effect of increasing the activity of the intestines is larger in the case of “propofol+DEX” than in the case of “midazolam+buprenorphine+propofol”.

In addition to this example, the peristaltic sound detection apparatus 10 can be used for measuring a change in the intestinal activity caused by oral ingestion of a medicine or a nutrient, a correlation between the blood sugar level and the intestinal activity, a correlation between cytokines and the intestinal activity, and the like.

(Supplementary Notes)

Each of the above-described blocks of the peristaltic sound detection apparatus 10 may be realized as hardware by a logical circuit formed on an integrated circuit (IC chip) or as software by a CPU (central processing unit).

In the latter case, the apparatus includes the CPU that executes commands issued by programs for realizing functions, a ROM (read-only memory) storing the programs, a RAM (random-access memory) in which the programs are expanded, and a storage device (recording medium) storing the programs and various pieces of data, such as a memory. An object of the present invention can be achieved even by supplying a recording medium on which program codes of control programs (executable programs, intermediate code programs, and source programs) of the apparatus, which are software that realizes the above-described functions, are recorded in a computer-readable manner to the apparatus and reading and executing the program codes recorded on the recording medium using a computer (or the CPU or an MPU).

As the recording medium, for example, a tape such as a magnetic tape or a cassette tape, one of disks including magnetic disks such as a floppy (registered trademark) disk and a hard disk and optical discs such as a CD-ROM, an MO, an MD, a DVD, and a CD-R, a card such as an IC card (includes a memory card) or an optical card, a semiconductor memory such as a mask ROM, an EPROM, an EEPROM (registered trademark), or a flash ROM, a logical circuit such as a PLD (programmable logic device) or an FPGA (field-programmable gate array), or the like may be used.

In addition, the apparatus may be configured to be connectable to a communication network, and the program codes may be supplied through the communication network. It is only required that the communication network be capable of transmitting the program codes, and the type thereof is not particularly limited. For example, the Internet, an intranet, an extranet, a LAN, ISDN, a VAN, a CATV communication network, a virtual private network, a telephone line network, a mobile communication network, a satellite communication network, or the like may be used. In addition, it is only required that a transmission medium configuring the communication network be a medium capable of transmitting the program codes, and the configuration and the type thereof are not particularly limited. For example, a wired medium such as IEEE 1394, a USB, a power-line carrier, a cable TV line, a telephone line, or an ADSL (asymmetric digital subscriber line) or a wireless medium such as infrared radiation as in IrDA or a remote control, Bluetooth (registered trademark), IEEE 802.11 wireless, HDR (high data rate), NFC (near field communication), DLNA (digital living network alliance), a mobile phone network, a satellite link, or a digital terrestrial network may be used.

The present invention is not limited to the above-described embodiments, and may be modified in various ways within the scope defined in the claims. Embodiments obtained by appropriately combining technological means disclosed in different embodiments are also included in the technical scope of the present invention.

The specific embodiments and examples described in the Description of Embodiments section are merely intended to clarify the technical content of the present invention, and the present invention is not to be limited to these specific examples and narrowly interpreted. The present invention may be modified and implemented in various ways within the spirit of the present invention and the claims, which are provided below.

### SUMMARY

A peristaltic sound detection apparatus according to a first aspect of the present invention includes biological sound detection means for detecting a biological sound emitted by intestines, frequency spectrum calculation means for calculating a frequency spectrum of the biological sound, matching coefficient calculation means for calculating a plurality of matching coefficients by individually matching the frequency spectrum of the biological sound and a plurality of standard frequency spectra of peristaltic sounds, and peristaltic sound determination means for determining whether or not the biological sound is a peristaltic sound by performing arithmetic processing on the plurality of matching coefficients.

According to the peristaltic sound detection apparatus configured in the above-described manner, a biological sound emitted by the intestines is detected through the biological sound detection means. The frequency spectrum calculation means calculates the frequency spectrum of the biological sound from the biological sound. The matching coefficient calculation means calculates matching coefficients by matching the frequency spectrum of the biological sound and standard frequency spectra. At this time, there are a plurality of standard frequency spectra, and the frequency spectrum of the biological sound is matched with each of the standard frequency spectra. Therefore, a plurality of matching coefficients are calculated from the frequency spectrum of the biological sound and the standard frequency spectra.

Thus, by calculating the plurality of matching coefficients, whether or not the biological sound is a peristaltic sound can be determined while detecting elements of various peristaltic sounds included in the biological sound. In addition, the peristaltic sound calculation means, the matching coefficient calculation, and the matching coefficient determination means do not require complex arithmetic processing.

Therefore, whether or not a biological sound is a peristaltic sound can be accurately determined without performing complex arithmetic processing. In other words, a peristaltic sound can be accurately detected without performing complex arithmetic processing.

In a peristaltic sound detection apparatus according to a second aspect of the present invention, it is preferable in the first aspect that the plurality of standard frequency spectra of peristaltic sounds are each a frequency spectrum of a peristaltic sound that occurs in a particular occurrence mode.

The plurality of standard frequency spectra are frequency spectra of peristaltic sounds caused by peristaltic movement in a plurality of occurrence modes. Therefore, the peristaltic sound detection apparatus according to the aspect of the present invention can detect elements of peristaltic sounds from a biological sound even if the biological sound includes elements of peristaltic sounds that occur in the plurality of occurrence modes.

In a peristaltic sound detection apparatus according to a third aspect of the present invention, the peristaltic sound determination means in the first or second aspect may be configured to, if a largest one of the plurality of matching coefficients is larger than a certain threshold, determine that the biological sound is a peristaltic sound.

According to the above configuration, if the largest one of the plurality of matching coefficients is larger than the certain threshold, the peristaltic sound determination means determines that the biological sound is a peristaltic sound. Therefore, the peristaltic sound detection apparatus according to the aspect of the present invention can accurately determine whether or not a biological sound is a peristaltic sound even if the biological sound includes elements of peristaltic sounds that occur in a plurality of occurrence modes.

In a peristaltic sound detection apparatus according to a fourth aspect, the peristaltic sound determination means in the first or second aspect may be configured to, if a value obtained by summing all the plurality of matching coefficients is larger than a certain threshold, determine that the biological sound is a peristaltic sound.

According to the above configuration, if the value obtained by summing the plurality of matching coefficients is larger than the certain threshold, the peristaltic sound determination means determines that the biological sound is a peristaltic sound. Therefore, the peristaltic sound detection apparatus according to the aspect of the present invention can accurately determine whether or not a biological sound is a peristaltic sound even if the biological sound includes little bits of elements in a plurality of occurrence modes.

In addition, a program for causing a computer to operate as the means included in the peristaltic sound detection apparatus according to each aspect of the present invention and a computer-readable recording medium on which the program is recorded are also included in the scope of the present invention.

A method for detecting a peristaltic sound according to a seventh aspect of the present invention includes a biological sound detection step of detecting a biological sound emitted by intestines, a frequency spectrum calculation step of calculating a frequency spectrum of the biological sound, a matching coefficient calculation step of calculating a plurality of matching coefficients by individually matching the frequency spectrum of the biological sound and a plurality of standard frequency spectra of peristaltic sounds, and a peristaltic sound determination step of determining whether or not the biological sound is a peristaltic sound by performing arithmetic processing on the plurality of matching coefficients.

According to the method for detecting a peristaltic sound configured in the above-described manner, the same advantageous effects as those produced by the peristaltic sound detection apparatus according to the first aspect can be produced.

### INDUSTRIAL APPLICABILITY

The present invention can be used as a peristaltic sound detection apparatus and a method for detecting a peristaltic sound that determine whether or not a sound emitted by the intestines is a peristaltic sound.

### REFERENCE SIGNS LIST

10 peristaltic sound detection apparatus

11 acoustic sensor (biological sound detection means)

12 frequency spectrum calculation unit (frequency spectrum calculation means)

13 matching coefficient calculation unit (matching coefficient calculation means)

14 storage unit

15 peristaltic sound determination unit (peristaltic sound determination means)

1-7. (canceled)

8. A peristaltic sound detection apparatus comprising:
biological sound detection means for detecting a biological sound emitted by intestines;
frequency spectrum calculation means for calculating a frequency spectrum of the biological sound;
matching coefficient calculation means for calculating a plurality of matching coefficients by individually matching the frequency spectrum of the biological sound and a plurality of standard frequency spectra of peristaltic sounds; and
peristaltic sound determination means for determining whether or not the biological sound is a peristaltic sound by performing arithmetic processing on the plurality of matching coefficients.

9. The peristaltic sound detection apparatus according to claim 8,
wherein the plurality of standard frequency spectra of peristaltic sounds are each a frequency spectrum of a peristaltic sound that occurs in a particular occurrence mode.

10. The peristaltic sound detection apparatus according to claim 8,
wherein, if a largest one of the plurality of matching coefficients is larger than a certain threshold, the peristaltic sound determination means determines that the biological sound is a peristaltic sound.

11. The peristaltic sound detection apparatus according to claim 9,
wherein, if a largest one of the plurality of matching coefficients is larger than a certain threshold, the peristaltic sound determination means determines that the biological sound is a peristaltic sound.

12. The peristaltic sound detection apparatus according to claim 8,
wherein, if a value obtained by summing all the plurality of matching coefficients is larger than a certain threshold, the peristaltic sound determination means determines that the biological sound is a peristaltic sound.

13. The peristaltic sound detection apparatus according to claim 9,
wherein, if a value obtained by summing all the plurality of matching coefficients is larger than a certain threshold, the peristaltic sound determination means determines that the biological sound is a peristaltic sound.

14. A non-temporary computer-readable recording medium on which a program for causing the peristaltic sound detection apparatus according to claim 8 to operate is recorded, the program causing a computer to function as the above-described means.

15. A non-temporary computer-readable recording medium on which a program for causing the peristaltic sound detection apparatus according to claim 9 to operate is recorded, the program causing a computer to function as the above-described means.

16. A non-temporary computer-readable recording medium on which a program for causing the peristaltic sound detection apparatus according to claim 10 to operate is recorded, the program causing a computer to function as the above-described means.

17. A non-temporary computer-readable recording medium on which a program for causing the peristaltic sound detection apparatus according to claim 11 to operate is recorded, the program causing a computer to function as the above-described means.

18. A method for detecting a peristaltic sound, the method comprising:
a biological sound detection step of detecting a biological sound emitted by intestines;
a frequency spectrum calculation step of calculating a frequency spectrum of the biological sound;
a matching coefficient calculation step of calculating a plurality of matching coefficients by individually matching the frequency spectrum of the biological sound and a plurality of standard frequency spectra of peristaltic sounds; and
a peristaltic sound determination step of determining whether or not the biological sound is a peristaltic sound by performing arithmetic processing on the plurality of matching coefficients.
